(19) United States
(12) Patent Application Publication
(10) Pub. No.: US 2004/0023822 A1
(43) Pub. Date: Feb. 5, 2004

(54) Surface-active compositions

(76) Inventors: Dietmar Ochs, Schopfheim (DE); Francois Brugger, Waltenheim (FR); Marcel Schnyder, Birsfelden (CH)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/450,226

(22) PCT Filed: Dec. 6, 2001
(86) PCT No.: PCT/EP01/14356

(30) Foreign Application Priority Data
Dec. 14, 2000 (EP) 00811192.4

(51) Int. Cl. C11D 1/00; C11D 9/50; A61K 7/50

(52) U.S. Cl. 510/218; 510/235; 510/276; 510/385; 510/386; 510/130

(57) ABSTRACT

A surface-active composition is described which comprises (a) from 0.01 to 90% by weight of a muixture of a microbicidal active ingredient of (a₁) a diphenyl ether compound and (a₂) a phenol derivative, (b) from 0 to 50% by weight of one or more hydrotropic agents, (c) from 0 to 80% by weight of one or more synthetic detergents or of a soap or of combinations of the mentioned substances and/or of a salt of a saturated and/or unsaturated C₈-C₂₂ fatty acid, (d) from 0 to 50% by weight of an alcohol, (e) from 0 to 50% by weightof typical ingredients for cleaning and disinfectant compositions and optionally (f) tap water or deionised water ad 10%. The compositions are used for the antimicrobial treatment of human skcin, and hair, of hard surfaces and of textile fibre materials.

The present invention relates to surface-active compositions and to the use of such compositions for the antimicrobial treatment of human skin and hair and for the treatment of hard surfaces and textile fibre materials.

Cleaning and disinfectant compositions comprising antimicrobial active ingredients, e.g. personal care preparations, hand and machine dishwashing formulations, cleaning and disinfecting formulations for hard surfaces and liquid and solid textile washing formulations, are becoming ever more widespread. Phenol derivatives and diphenyl ether compounds are known as antibacterial active ingredients.

It has now been found, surprisingly, that a combination of diphenyl ether compounds and phenol derivatives exhibits strong bactericidal effects.

The present invention accordingly relates to a surface-active composition comprising

(a) from 0.01 to 90% by weight of a mixture of a microbicidal active ingredient of (a₁) a diphenyl ether compound and (a₂) a phenol derivative,

(b) from 0 to 50% by weight of one or more hydrotropic agents,

(c) from 0 to 80% by weight of one or more synthetic detergents or of a soap or of combinations of the mentioned substances and/or of a salt of a saturated and/or unsaturated C₈-C₂₂ fatty acid,

(d) from 0 to 50% by weight of an alcohol,

(e) from 0 to 50% by weight of typical ingredients for cleaning and disinfectant compositions and optionally,

(f) tap water or deionised water ad 100%.

Preferably, the present invention accordingly relates to a surface-active composition comprising

(a) from 0.01 to 10% by weight of a mixture of a microbicidal active ingredient of (a₁) a diphenyl ether compound and (a₂) a phenol derivative,

(b) from 0 to 50% by weight of one or more hydrotropic agents,

(c) from 5 to 80% by weight of one or more synthetic detergents or of a soap or of combinations of the mentioned substances and/or of a salt of a saturated and/or unsaturated C₈-C₂₂ fatty acid,

(d) from 0 to 50% by weight of an alcohol, and optionally

(f) tap water or deionisied water ad 100%.

The composition according to the invention preferably comprises as component (a₁) a hydroxy-diphenyl ether of formula

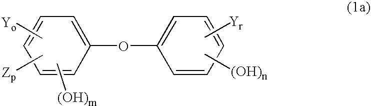

wherein

Y is chlorine or bromine,

Z is SO₂H, NO₂ or C₁-C₄alkyl,

r is from0 to 3,

o is from 0 to 3,

p is 0, 1 or 2,

m is 1 or 2 and

n is 0 or 1,

and more especially a compound of

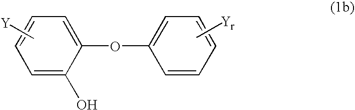

wherein

Y is chlorine and

r is 1 or2.

Very special preference is given to a compound of formula

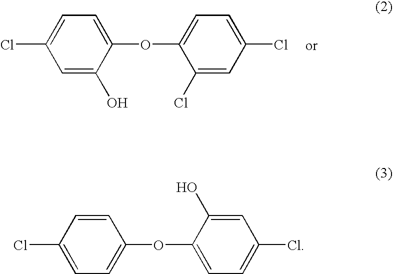

As component (a₁) there can also be used a non-halogenated hydroxydiphenyl ether of formula

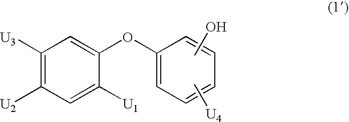

wherein

U₁ and U₂ are each independently of the other hydrogen, hydroxy, unsubstituted or hydroxy-substituted C₁-C₂₀alkyl, C₅-C₇cycloalkyl, C₁-C₆alkylcarbonyl, C₁-C₂₀alkoxy, phenyl or phenyl-C₁-C₃alkyl;

U₃ is hydrogen, C₁-C₂₀alkyl, C₁-C₂₀alkoxy or C₁-C₆alkylcarbonyl; and

U₄ is hydrogen, unsubstituted or hydroxy-substituted C₁-C₂₀alkyl, C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl.

U₁, U₂, U₃ and U₄ having the meaning of C₁-C₂₀alkyl are straight-chain or branched alkyl radicals, for example methyl, ethyl, n-propyl, isopropyl, n-butyl, sec-butyl, tert-butyl, pentyl, isopentyl, tert-pentyl, hexyl, cyclohexyl, heptyl, octyl, isooctyl, nonyl, decyl and the like.

U₁, U₂ and U₃ as C₁-C₂₀alkoxy are straight-chain or branched alkoxy radicals, for example methoxy, ethoxy, n-propoxy, isopropoxy, n-butoxy, sec-butoxy, tert-butoxy, pentyloxy, iso-pentyloxy, tert-pentyloxy, heptyloxy, octyloxy, isooctyloxy, nonyloxy, decyloxy and the like.

U₁, U₂, U₃ and U₄ having the meaning of C₁-C₆alkylcarbonyl are straight-chain or branched carbonyl radicals, for example acetyl, propionyl, butyryl, isobutyryl, valeryl, isovaleryl, pivaloyl and the like.

U₁, U₂ and U₄ having the meaning of hydroxy-substituted C₁-C₂₀alkyl are, for example, hydroxymethyl, hydroxyethyl, hydroxypropyl, hydroxybutyl, hydroxypentyl, hydroxyhexyl, hydroxyheptyl, hydroxyoctyl, hydroxynonyl, hydroxydecyl and the like.

According to the invention, preference is given to the use of compounds of formula (1′) wherein OH is in the meta- or para-position relative to the ether bond.

Preferably, U₁ and U₂ are each independently of the other hydrogen, C₁-C₂₀alkyl, C₁-C₆alkyl-carbon or C₁-C₂₀alkoxy.

U₃ is preferably hydrogen, C₁-C₂₀alkyl or C₁-C₂₀alkoxy.

U₄ is preferably hydrogen, C₁-C₂₀alkyl, hydroxy, formyl, acetonyl, allyl, carboxymethyl, carboxyallyl, hydroxy-substituted C₁-C₂₀alkyl or C₁-C₆alkylcarbonyl.

Compounds of formula (1′) that are of special interest are:

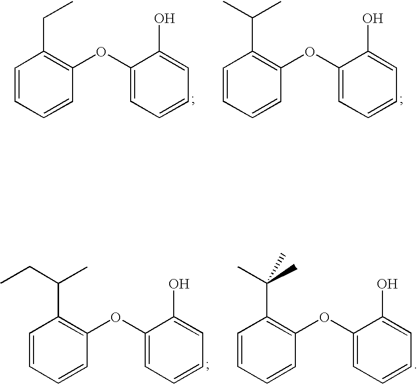

Other compounds of formula (1′) that are of special interest are:

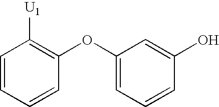

wherein

U₁ is C₁-C₅alkyl, for example the compound of formula

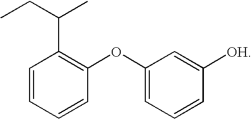

Also of interest are compounds of formula

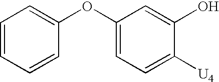

wherein

U₄ is C₁-C₅alkyl, for example the compound of formula

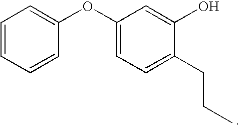

The following compounds are of special interest:

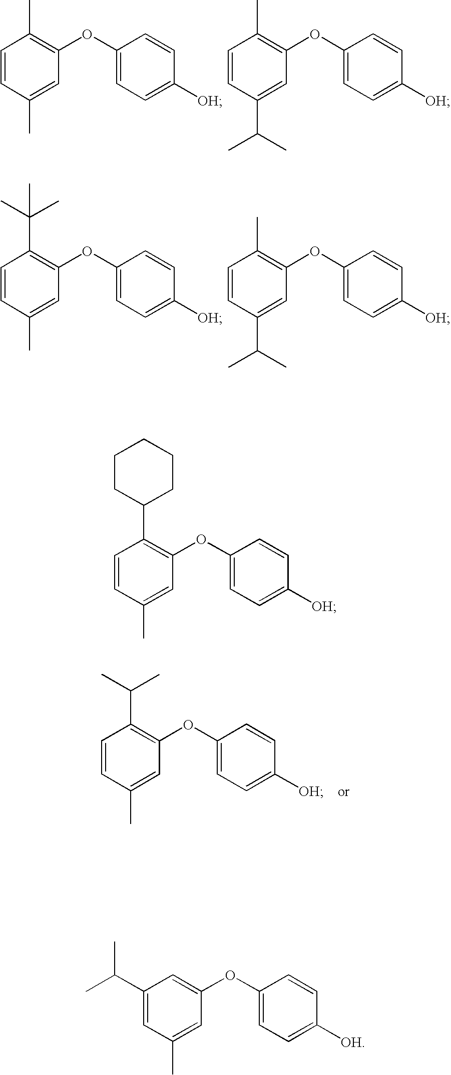

The compounds of formula (1′) are known or can be prepared using methods analogous to those known.

Compounds suitable as component (a₂) are preferably those selected from phenol derivatives of formula

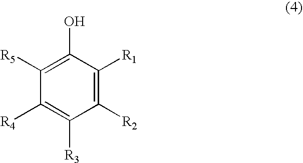

wherein

R₁ is hydrogen, hydroxy, C₁-C₂₀alkyl, chloro, phenyl, benzyl or nitro,

R₂ is hydrogen, hydroxy, C₁-C₂₀alkyl or halogen,

R₃ is hydrogen, C₁-C₂₀alkyl, hydroxy or chloro,

R₄ is hydrogen or methyl, and

R₅ is hydrogen or nitro.

Exemplary compounds are chlorophenols (o-, m-, p-chlorophenols), 2,4-dichlorophenol, p-nitrophenol, xylenol, p-chloro-m-xylenol, cresols (o-, m-, p-cresols), p-chloro-m-cresol, pyrocatechol, resorcinol, orcinol, 4-n-hexylresorcinol, pyrogallol, phloroglucinol, carvacrol, thymol, p-chlorothymol, o-phenylphenol, o-benzylphenol and p-chloro-o-benzylphenol.

Further exemplary representatives of component (a₂) are chlorhexidines, for example 1,1′-hexamethylene-bis (5-(p-chlorophenyl)biguanide), together with organic and inorganic acids and chlorhexidine derivatives, such as their diacetates, digluconates or dihydrochloride compounds.

Further exemplary phenol derivatives are 1-phenoxypropan-2-ol and 3-(4-chlorophenoxy)-1,2-propanediol.

Very special preference is given to the use of o-phenylphenol as component (a₂).

In the composition according to the invention, the combination of (a₁) the compound of formula (2) or (3) and (a₂) o-phenylphenol is especially used.

The following compounds are suitable as component (b):

sulfonates of terpenoids, or of mono- or di-nuclear aromatic compounds, for example sulfonates of camphor, toluene, xylene, cumene or of naphthol;

saturated or unsaturated C₃-C₁₂ di- or poly-carboxylic acids, for example malonic, succinic, glutaric, adipic, pimelic, suberic, azelaic and sebacic acid, undecane- and dodecane-dioic acid, fumaric, maleic, tartaric and malic acid, and citric and aconitic acid;

aminocarboxylic acids, such as ethylenediaminetetraacetic acid, hydroxyethylethylene-diaminetetraacetic acid and nitrilotriacetic acid;

cycloaliphatic carboxylic acids, such as camphoric acid;

aromatic carboxylic acids, such as benzoic, phenylacetic, phenoxyacetic and cinnamic acid, 2-, 3- and 4-hydroxybenzoic acid, anilic acid, and o-, m- and p-chlorophenylacetic acid and o-, m- and p-chlorophenoxyacetic acid;

isethionic acid;

tannic acid;

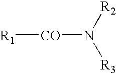

acid amides of formula

wherein

R₁ is hydrogen or C₁-C₁₂alkyl and

R₂ and R₃ are each independently of the other hydrogen, C₁-C₁₂alkyl, C₂-C₁₂alkenyl, C₂-C₁₂hydroxyalkenyl, C₂-C₁₂hydroxyalkyl or a polyglycol ether chain having from 1 to 30 —CH₂—CH₂—O— or —CHY₁—CHY₂—O— groupings, wherein one of the radicals Y₁ and Y ₂ is hydrogen and the other is methyl, such as N-methylacetamide;

urea derivatives of formula

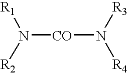

wherein

R₁, R₂, R₃ and R₄ are each independently of the others hydrogen, C₁-C₈alkyl, C₂-C₈alkenyl, C₁-C₈hydroxyalkyl or C₂-C₈hydroxyalkenyl.

All the organic acids mentioned under (b) can also be in the form of their water-soluble salts, such as the alkali metal salts, especially the sodium or potassium salts, or the amine (NR₁R₂R₃) salts wherein

R₁, R₂ and R₃ are each independently of the others hydrogen, C₁-C₈alkyl, C₂-C₈alkenyl, C₁-C₈-hydroxyalkyl, C₅-C₈cycloalkyl or polyalkyleneoxy-C₁-C₁₈alkyl or

R₁, R₂ and R₃, together with the nitrogen atom to which they are bonded, are unsubstituted or C₁-C₄alkyl-substituted morpholino.

Component (b) can consist of a single compound or a plurality of different compounds.

Very special preference is given to a combination of cumenesulfonate and citric acid monohydrate.

As component (c), anionic, nonionic, or zwitterionic and amphoteric synthetic detergents are suitable.

Suitable anionic detergents are

sulfates, for example fatty alcohol sulfates, the alkyl chain of which has from 8 to 18 carbon atoms, for example sulfated lauryl alcohol;

fatty alcohol ether sulfates, for example the acid esters or salts thereof of a polyaddition product of from 2 to 30 mol of ethylene oxide and 1 mol of a C₈-C₂₂ fatty alcohol;

the alkali metal, ammonium or amine salts, referred to as soaps, of C₈-C₂₀ fatty acids, for example coconut fatty acid;

alkylamide sulfates;

alkylamine sulfates, for example monoethanolamine lauryl sulfate;

alkylamide ether sulfates;

alkylaryl polyether sulfates;

monoglyceride sulfates;

alkanesulfonates, the alkyl chain of which contains from 8 to 20 carbon atoms, for example dodecyl sulfonate;

alkylamide sulfonates;

alkylaryl sulfonates;

α-olefin sulfonates;

sulfosuccinic acid derivatives, for example alkyl sulfosuccinates, alkyl ether sulfo-succinates or alkylsulfosuccinamide derivatives;

N-[alkylamidoalkyl]amino acids of formula

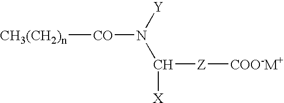

wherein

X is hydrogen, C₁-C₄alkyl or —COO⁻M⁺,

Y is hydrogen or C₁-C₄alkyl, —(CH₂)—

Z is m₁−1

m₁ is from 1 to 5,

n₁ is an integer from 6 to 18 and

M is an alkali metal cation or amine cation,

alkyl and alkylaryl ether carboxylates of formula (13) CH₃—X—Y-A wherein

X is a radical of formula —(CH₂)₅₋₁₉—O—,

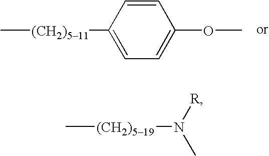

R is hydrogen or C₁-C₄alkyl,

Y is —(CHCHO)₁₋₅₀—,

A is (CH₂)ₘ₂₋₁—COO⁻M⁺ or

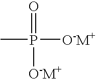

m₂ is from 1 to 6 and

M is an alkali metal cation or amine cation.

Also used as anionic surfactants are fatty acid methyl taurides, alkyl isothionates, fatty acid polypeptide condensation products and fatty alcohol phosphoric acid esters. The alkyl radicals occurring in those compounds preferably have from 8 to 24 carbon atoms.

The anionic surfactants are generally in the form of their water-soluble salts, such as the alkali metal, ammonium or amine salts. Examples of such salts include lithium, sodium, potassium, ammonium, triethylamine, ethanolamine, diethanolamine and triethanolamine salts. The sodium, potassium or ammonium (NR₁R₂R₃) salts, especially, are used, with R₁, R₂ and R₃ each independently of the others being hydrogen, C₁-C₄alkyl or C₁-C₄hydroxyalkyl.

Especially preferred anionic surfactants in the composition according to the invention are monoethanolamine lauryl sulfate or the alkali metal salts of fatty alcohol sulfates, especially sodium lauryl sulfate and the reaction product of from 2 to 4 mol of ethylene oxide and sodium lauryl ether sulfate.

Suitable zwitterionic and amphoteric surfactants include C₈-C₁₈betaines, C₈-C₁₈sulfobetaines, C₈-C₂₄alkylamido-C₁-C₄alkylenebetaines, imidazoline carboxylates, alkylamphocarboxy-carboxylic acids, alkylamphocarboxylic acids (e.g. lauroamphoglycinate) and N-alkyl-β-aminopropionates or -iminodipropionates, with preference being given to C₁₀-C₂₀alkylamido-C₁-C₄akylenebetaines and especially to coconut fatty acid amide propylbetaine.

Nonionic surfactants that may be mentioned include, for example, derivatives of the adducts of propylene oxide/ethylene oxide having a molecular weight of from 1000 to 15 000, fatty alcohol ethoxylates (1-50 EO), alkylphenol polyglycol ethers (1-50 EO), polyglucosides, ethoxylated hydrocarbons, fatty acid glycol partial esters, for example diethylene glycol monostearate, fatty acid alkanolamides and dialkanolamides, fatty acid alkanolamide ethoxylates and fatty amine oxides.

As component (c) there may also be used the salts of saturated and unsaturated C₈-C₂₂ fatty acids either alone or in the form of a mixture with one another or in the form of a mixture with other detergents mentioned as component (c). Examples of such fatty acids include, for example, capric, lauric, myristic, palmitic, stearic, arachidic, behenic, caproleic, dodecenoic, tetradecenoic, octadecenoic, oleic, eicosenoic and erucic acid, and the commercial mixtures of such acids, such as, for example, coconut fatty acid. Such acids are present in the form of salts, there coming into consideration as cations alkali metal cations, such as sodium and potassium cations, metal atoms, such as zinc and aluminium atoms, and nitrogen-containing organic compounds of sufficient alkalinity, such as amines and ethoxylated amines. Such salts may also be prepared in situ.

As component (d) there come into consideration as dihydric alcohols especially those compounds having from 2 to 6 carbon atoms in the alkylene moiety, such as ethylene glycol, 1,2- or 1,3-propanediol, 1,3-, 1,4- or 2,3-butanediol, 1,5-pentanediol and 1,6-hexanediol.

Preference is given to 1,2-propanediol (propylene glycol).

Preferred monohydric alcohols are ethanol, n-propanol and isopropanol and mixtures of those alcohols.

The composition according to the invention comprises, as component (e), builders (zeolites/layered silicates), bleaching agents or bleaching systems (perborate/percarbonate plus TAED), fluorescent whitening agents and enzymes.

Furthermore, the washing composition can comprise enzymes, enzyme stabilisers, thickeners, sequestering agents, for example EDTA or phosphoric acid salts, corrosion inhibitors, colourants, perfumes, fluorescent whitening agents, buffer compounds or the like.

Compositions according to the invention can be prepared by mixing components (a) and optionally (b), (c), (d) and (e) in any desired order with the requisite amount of deionised water and stirring the batch until homogeneous. The composition is made up to 100% with tap water or deionised water. The procedure is purely physical. No chemical reaction takes place between the individual components.

Cleaning and disinfecting formulations according to the present invention may further comprise thickening agents, sequestering agents, antioxidants, UV absorbers, dyes, perfumes, buffer compounds, vitamins, moisturizers, body care substances, solids like waxes etc.

The formulations according to the invention exhibit strong bactericidal activity in two respects:

rapid destruction of germs present.

This can be demonstrated, for example, by a suspension test, e.g. according to test method EN 1276.

long-term bactericidal activity on the treated surface, as a result of which recolonisation is prevented effectively.

This can be demonstrated, for example, by the AATCC 100-1993 method.

They are therefore suitable for disinfecting and cleaning human skin and hands, hard articles and textile fibre materials and can be applied thereto in dilute or undiluted form, an amount of at least 2 ml, preferably in the undiluted form, being suitable for disinfection of the hands.

The compositions according to the invention are very especially used in washing and cleaning formulations, for example in household washing formulations, powder washing formulations, washing pastes, fabric softeners, solid soaps, dishwashing formulations, all-purpose cleaners, especially in liquid washing formulations for textile fibre materials.

The invention accordingly relates also to a method for the antimicrobial treatment of textile fibre materials in washing liquor, which method comprises treating the textile fibre materials in the washing liquor with a composition comprising

(a) a microbicidal active ingredient of

(a₁) from 0 to 5% by weight of a diphenyl ether compound and

(a₂) from 0.1 to 5% by weight of a phenol derivative,

(b) from 0 to 50% by weight of one or more hydrotropic agents,

(c) from 5 to 80% by weight of one or more synthetic detergents or of a soap or of combinations of the mentioned substances and/or of a salt of a saturated and/or unsaturated C₈-C₂₂fatty acid,

(f) from 0 to 50% by weight of an alcohol, and optionally

(e) from 0 to 50% by weight of typical ingredients for cleaning and disinfectant compositions and optionally

(f) tap water or deionised water ad 100%.

In the method according to the invention preference is given to a washing liquor that is free of diphenyl ether compounds, that is to say contains no component (a₁).

The invention relates also to a method for imparting antimicrobial properties to textile fibre materials, which method comprises treating the textile fibre materials in the washing liquor with a composition comprising

(a) from 0.01 to 10% by weight of a mixture of a microbicidal active ingredient of

(a₁) a diphenyl ether compound and

(a₂) a phenol derivative,

(b) from 0 to 50% by weight of one or more hydrotropic agents,

(c) from 5 to 80% by weight of one or more synthetic detergents or of a soap or of combinations of the mentioned substances and/or of a salt of a saturated and/or unsaturated C₈-C₂₂ fatty acid,

(g) from 0 to 50% by weight of an alcohol,

(h) from 0 to 50% by weight of typical ingredients for cleaning and disinfectant compositions and optionally and optionally

(i) tap water or deionised water ad 100%,

at least a fraction of the antimicrobial active ingredient remaining on the textile fibre material.

The textile materials that can be treated in accordance with the invention are undyed or dyed or printed, natural or synthetic fibre materials, for example of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose. Preferred suitable textile fibre materials are of cotton.

Using the composition according to the invention it is possible to destroy bacteria present on the washing material in the dilute liquor during the washing procedure. At the same time, antimicrobial properties are imparted to the washed textile material, that is to say bacteria that get on the textile material while it is being worn are destroyed.

The following Examples illustrate the invention. Percentages and parts are percentages by weight and parts by weight, respectively.

### EXAMPLES 1

Preparation of a Liquid Washing Formulations (1-(5)

Liquid formulations having the following compositions are prepared:

| Formulation | 1 | 2 | 3 | 4 | 5 |
|---|---|---|---|---|---|
| combination of 30% of the | 0.6 | 0.6 | 0.6 | — | — |
| compound of formula (3) and 70% of | | | | | |
| propylene glycol | | | | | |
| o-phenylphenol | 0.5 | 1 | 1 | 1 | 2 |
| sodium dodecylbenzenesulfonate | 6 | 6 | 6 | 6 | 6 |
| sodium lauryl sulfate | 8 | 8 | 8 | 8 | 8 |
| Pareth 45-7 (Dobanol 45-7) | 4 | 4 | 4 | 4 | 4 |
| ethanol | 9 | 9 | 9 | 9 | 9 |
| sodium cumenesulfonate | 5 | − | 5 | 5 | − |
| soap noodles (Mettler) | 5 | 7 | 7 | 5 | 7 |
| trisodium citrate dihydrate | 2 | 2 | 2 | 2 | 2 |
| triethanolamine | 5 | 5 | 5 | 5 | 5 |
| fluorescent whitening agents | 0.3 | 0.3 | 0.3 | 0.3 | 0.3 |
| water to | 100 | 100 | 100 | 100 | 100 |

### EXAMPLE 2

Determination of the Bactericidal Efficacy of Formulations (1) to (5) in Accordance with EN 1276 (Concentration 80%, Contact Time 5 Minutes) in Log Reduction

Test Principle:

1.0 ml of a bacterial suspension is added to 8.0 ml of the formulation in question (the test concentration is multiplied by a factor of 1.25) and to 1.0 ml of a suspension of 0.3% (factor 10) bovine albumin and mixed vigorously. After the contact time (see above) at 21° C. (±1° C.), a 0.1 ml sample is removed and added to 50 ml of TSB+inactivator (=test neutralisation mixture, 10°). 500 μl of the neutralisation mixture are added to 9 ml of TSB+inactivator to give a 10⁻² dilution. Each test neutralisation mixture and the dilutions are filtered over a membrane and washed with 150 ml of distilled water. The membranes are incubated for 48 hours on the surface of agar plates. After incubation, the colonies are counted and listed in a Table, and the log reduction is calculated.

The results are given in Table 1.

TABLE 1

| Formulation | 1 | 2 | 3 | 4 | 5 |
|---|---|---|---|---|---|
| S.a. ATCC 6538 | >5 | >5 | 4.6 | 4.5 | >5 |
| E.c. ATCC 10536 | >5 | >5 | 4.5 | 3.8 | >5 |
| E.h. ATCC 10541 | >5 | >5 | >5 | >5 | >5 |
| Ps.a. ATCC 15442 | 3 | 4.3 | 4 | 3.9 | >5 |

The results in Table 1 show that good bactericidal effects can be achieved on the textile material using the formulations according to the invention.

### EXAMPLE 3

Determination of the Bactericidal Effects, During Wear (from 0 to 24 Hours), on Textile Material Washed Under Standard Conditions

Textile material: cotton

| | |
|---|---|
| washing formulation: | 2.3 g |
| water: | 300 ml |
| liquor ratio: | 1:10 |
| duration of treatment: | 10 min |
| temperature: | 40° C. |

Test Principle:

Round cotton textile patches which have been washed under standard conditions (2.3 g detergent in a 300 ml liquor; 30 g textile; washing period: 10 minutes at 40° C.) are placed in sterile Petri dishes (diameter: 55 mm).

All the samples are then inoculated with 0.25 ml of a bacterial suspension (approx. ˜10⁵ cfu/sample) and placed in a humidity chamber at 37° C.

Directly after inoculation and after 8 and 24 hours at 37° C., the inoculated textile patches are placed in 50 ml of 0.07 molar phosphate buffer (pH 7.4, containing 1% Tween 80 and 0.3% lecithin) and shaken for 1 minute. After shaking, a dilution series in sterile distilled water, down to a concentration of 10⁻², is prepared. 100 μl samples of the undiluted solution and of the 10⁻¹ and 10⁻² dilutions are applied to the plates using a spiralometer. After incubation, the surviving colonies are counted, calculated as cfu/sample and given in Table 2 herein below.

| Formulation | | | |
|---|---|---|---|
| of the detergents | 6 | 7 | 8 |
| combination of 30% | — | — | 0.6 |
| of the compound of | | | |
| formula (3) and 70% | | | |
| of propylene glycol | | | |
| o-phenylphenol | — | 2 | 0.5 |
| sodium dode- | 20 | 20 | 20 |
| cylbenzenesulfonate | | | |
| Pareth 45-7 (Dobanol | 14 | 14 | 14 |
| 45-7) ethanol | 9 | 9 | 9 |
| soap noodles (Mettler) | 10 | 10 | 10 |
| trisodium citrate | 4 | 4 | 4 |
| dihydrate | | | |
| triethanolamine | 5 | 5 | 5 |
| water | ad 100% | ad 100% | ad 100% |
| pH “as is” | 10.5 | 10.3 | 10.3 |
| appearance | clear, yellowish | clear, yellowish | clear, yellowish |

TABLE 2
AATCC 100-1993
cotton washed with a washing formulation
under standard conditions (2.3 g of
det./300 ml of water/washing
liquor 1:10, 10 min., 40° C.)

| Formulation | | 6 | 7 | 8 |
|---|---|---|---|---|
| S. aureus ATCC 6538 | 0 h | 4.4 × 10⁵ | | |
| | | | 3.6 × 10⁵ | 4.1 × 10⁵ |
| | 8 h | | 3.9 × 10⁵ | |
| | | 8.2 × 10⁵ | | |
| | 24 h | 1.0 × 10⁷ | 4.1 × 10⁵ | <100 |
| Klebsiella pneumoniae | 0 h | 1.9 × 10⁵ | | |
| ATCC 4352 | | | 1.8 × 10⁵ | 1.8 × 10⁵ |
| | 8 h | 5.7 × 10⁸ | 4.3 × 10⁸ | 3.1 × 10² |
| | | | | 1 × 10² |
| | 24 h | 2.7 × 10⁹ | 1.6 × 10⁹ | <100 |

Only detergent compositions comprising the compound of formula (3) show a distinct antimicrobial activity on the textile material.

### EXAMPLE 4

Preparation of Further Liquid Washing Formulations

| | Formulation | | | | | | | | | | |
|---|---|---|---|---|---|---|---|---|---|---|---|
| Components | 9 | 10 | 11 | 12 | 13 | 14 | 15 | 16 | 17 | 18 | 19 |
| combination of 30% of the | 0.6 | 0.6 | 0.6 | 0.6 | 0.6 | 0.6 | 0.6 | 0.6 | 0.6 | 0.6 | 0.6 |
| compound of formula (3) and | | | | | | | | | | | |
| 70% of propylene glycol | | | | | | | | | | | |
| o-phenylphenol | 0.9 | 0.9 | 0.9 | 0.9 | 0.9 | 0.9 | 0.9 | 0.9 | 0.9 | 0.9 | 0.9 |
| dodecylbenzenesulfonic acid | 7.5 | | | | | | | 8.5 | | | |
| sodium | | 27 | 23.6 | 10 | 28 | | | | 20 | 24 | 6 |
| dodecylbenzenesulfonate | | | | | | | | | | | |
| sodium laureth sulfate 3 EO | 17 | 10 | | | | | | | | | |
| sodium lauryl sulfate | | | | | | 6 | | | | | 8 |
| coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ oxoalcohol | | | | 20 | 9 | 14.5 | 12 | 29 | 26 | | |
| PEG-8 C₁₃₋₁₅ fatty alcohol | | | | | | | 10 | | | | |
| alkyl polyglucoside | | | 5 | | | 1 | 2 | | | | |
| laureth-1 | | 5 | | | | | | | | | |
| PPG | | | | 2 | 3 | 8 | | | | | |
| sodium carbonate | | | 2 | | | | | | | | |
| sodium tripolyphosphate | | | 20 | | | | | | | | |
| potassium tripolyphosphate | | 22 | | | | | | | | | |
| 50% | | | | | | | | | | | |
| sodium cumenesulfonate 40% | | 25 | | | | | | | | | |
| trisodium citrate | 5.5 | | | | | 2 | | | | | 2 |
| lauryltrimonium chloride | 0.7 | | | | | | | | | | |
| polycarboxylate | | | | 13 | 18 | 15 | 10 | 23 | 16.2 | | |
| 2-propanol | 6 | | | 7 | 3 | | 4 | 9.5 | 8 | | |
| ethanol | 6 | | | | | | | | | 9 | |
| glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent whitening agent | 0.1 | 0.1 | 0.1 | 0.1 | 0.1 | 0.1 | 0.1 | | | | 0.1 |
| Tinopal CBS-x | | | | | | | | | | | |
| fluorescent whitening agent | | | | | | | | 0.1 | 0.1 | 0.1 | |
| Tinopal CBS-CL | | | | | | | | | | | |
| Soap | | | | | | | | | | | 7 |
| water to | 100 | 100 | 100 | 100 | 100 | 100 | 100 | 100 | 100 | 100 | |

### EXAMPLE 5

Preparation of Different Formulations:

| | formulation | | | | |
|---|---|---|---|---|---|
| components | 20 | 21 | 22 | 23 | 24 |
| combination of 30% of the | 0.13 | 0.13 | 0.6 | 0.6 | 0.3 |
| compound of formula (3) and | | | | | |
| 70% of propylene glycol | | | | | |
| o-phenylphenol | 0.2 | 0.2 | 0.9 | 0.9 | 0.45 |
| sodium laureth sulfate | 9.0 | 15 | 1.2 | | |
| cocamidopropyl betaine | 3.0 | 4.5 | 1 | | |
| decyl glucoside | 3.0 | | | | |
| citiric acid | 0.1 | 0.1 | | 3 | |
| polyquaternium-7 | | 0.4 | | | |
| lauramine oxide | | | 1 | | |
| sodium Citrate | | | 4 | | |
| sodium carbonate | | | 3 | | |
| ethanol | | | 3 | | |
| sodium C₁₄₋₁₇ alkyl sec. | | | | 16.6 | |
| sulfonate | | | | | |
| sodium laurylsulfate | | | | 20 | |
| Laureth-09 | | | | 3 | |
| sodium cumolsulfonate | | | | 5 | |
| sodium chloride | | | | 3 | |
| Quaternium 18 and | | | | | 4 |
| iospropylalcohol | | | | | |
| Pareth-25-7 | | | | | 0.5 |
| water to | 100 | 100 | 100 | 100 | 100 |

Formulation 20: shower gel
Formulation 21: shampoo
Formulation 22: all purpose cleaner
Formulation 23: dish washing detergent
Formulation 24: softener detergent

What is claimed is:

1. A surface-active composition according to claim 1 comprising
(a) from 0.01 to 90% by weight of a mixture of a microbicidal active ingredient of
(a₁) a diphenyl ether compound and
(a₂) a phenol derivative,
(b) from 0 to 50% by weight of one or more hydrotropic agents,
(c) from 0 to 80% by weight of one or more synthetic detergents or of a soap or of combinations of the mentioned substances and/or of a salt of a saturated and/or unsaturated C₈-C₂₂ fatty acid,
(d) from 0 to 50% by weight of an alcohol,
(e) from 0 to 50% by weight of typical ingredients for cleaning and disinfectant compositions and optionally
(f) tap water or deionised water ad 100%.

2. Composition according to claim 1 comprising
(a) from 0.01 to 10% by weight of a mixture of a microbicidal active ingredient of
(a₁) a diphenyl ether compound and
(a₂) a phenol derivative,
(b) from 0 to 50% by weight of one or more hydrotropic agents,
(c) from 5 to 80% by weight of one or more synthetic detergents or of a soap or of combinations of the mentioned substances and/or of a salt of a saturated and/or unsaturated C₈-C₂₂ fatty acid,
(d) from 0 to 50% by weight of an alcohol, and optionally
(e) tap water or deionised water ad 100%.

3. A composition according to claim 1 or 2, wherein there is used as component (a₁) a 2-hydroxy-diphenyl ether of formula 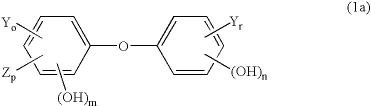
wherein
Y is chlorine or bromine,
Z is SO₂H, NO₂ or C₁-C₄alkyl,
r is from 0 to 3,
o is from 0 to 3,
p is 0, 1 or 2,
m is 1 or 2 and
n is 0 or 1.

4. A composition according to claim 3, wherein there is used a compound of formula 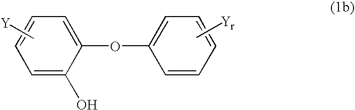
wherein
Y is chlorine and
r is 1 or 2.

5. A composition according to any one of claims 1 to 4, wherein there is used a 2-hydroxy-diphenyl ether of formula

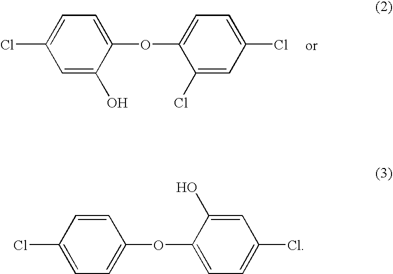

6. A composition according to claim 1 or 2, wherein there is used as component (a₁) a diphenyl ether compound of formula 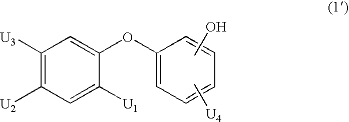
wherein
U₁ and U₂ are each independently of the other hydrogen, hydroxy, unsubstituted or hydroxy-substituted C₁-C₂₀alkyl; C₅-C₇cycloalkyl, C₁-C₆alkylcarbonyl, C₁-C₂₀alkoxy, phenyl or phenyl-C₁-C₃alkyl;
U₃ is hydrogen, C₁-C₂₀alkyl, C₁-C₂₀alkoxy or C₁-C₆alkylcarbonyl; and
U₄ is hydrogen, unsubstituted or hydroxy-substituted C₁-C₂₀alkyl; C₅-C₇cycloalkyl, hydroxy, formyl, acetonyl, C₁-C₆alkylcarbonyl, C₂-C₂₀alkenyl, carboxy, carboxy-C₁-C₃alkyl, C₁-C₃alkylcarbonyl-C₁-C₃alkyl or carboxyallyl.

7. A composition according to any one of claims 1 to 6, wherein there is used as component (a₂) a compound of formula 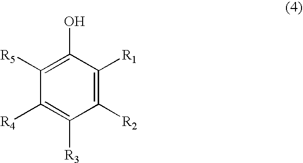
wherein
R₁ is hydrogen, hydroxy, C₁-C₂₀alkyl, chloro, phenyl, benzyl or nitro,
R₂ is hydrogen, hydroxy, C₁-C₂₀alkyl or halogen,
R₃ is hydrogen, C₁-C₂₀alkyl, hydroxy or chloro,
R₄ is hydrogen or methyl, and
R₅ is hydrogen or nitro.

8. A composition according to claim 7, wherein o-phenylphenol is used as component (a₂).

9. A composition according to any one of claims 1 to 8, wherein the compound of formula (2) or (3) is used as component (a₁) and o-phenylphenol is used as component (a₂).

10. A composition according to any one of claims 1 to 9, wherein a sulfonate of a terpenoid or of a mono- or di-nuclear aromatic compound is used as component (b).

11. A composition according to claim 9, wherein a mono- or di-nuclear aromatic compound the sulfonate of camphor, toluene, xylene, cumene or of naphthol is used as component (b).

12. A composition according to any one of claims 1 to 9, wherein a saturated or unsaturated C₃-C₁₂ di- or poly-carboxylic acid is used as component (b).

13. A composition according to either claim 10 or claim 11, wherein a combination of cumenesulfonate and citric acid monohydrate is used as component (b).

14. A composition according to any one of claims 1 to 121, wherein a C₁₀-C₂₀alkylamido-C₁-C₄alkylenebetaine is used as component (b).

15. A composition according to any one of claims 1 to 13, wherein a salt of lauric, myristic, palmitic, stearic, arachidic, behenic, caproleic, dodecenoic, tetradecenoic, octadecenoic, oleic, eicosenoic or erucic acid is used as component (c).

16. A composition according to any one of claims 1 to 154, wherein propylene glycol is used as component (d).

17. A composition according to any one of claims 1 to 15, wherein ethanol, propanol, isopropanol or a mixture of those alcohols is used as component (d).

18. Use of the composition according to any one of claims 1 to 17 for the antimicrobial treatment of human skin and hair.

19. Use of the composition according to any one of claims 1 to 17 for the antimicrobial treatment of hard surfaces.

20. Use according to claim 19, wherein the composition is used in dishwashing formulations.

21. Use according to claim 19, wherein the composition is used in all-purpose cleaners.

22. Use of the composition according to any one of claims 1 to 17 for the antimicrobial treatment of textile fibre materials.

23. Use according to claim 22, wherein the composition is used in powder washing formulations, washing pastes, liquid washing formulations, fabric softeners or solid soaps.

24. A method for the antimicrobial treatment of textile fibre materials in washing liquor, which method comprises treating the textile fibre materials in the washing liquor with a composition comprising
(a) a microbicidal active ingredient of
(a₁) from 0 to 5% by weight of a diphenyl ether compound and
(a₂) from 0.1 to 5% by weight of a phenol derivat
(b) from 0 to 50% by weight of one or more hydrotropic agents,
(c) from 5 to 80% by weight of one or more synthetic detergents or of a soap or of combinations of the mentioned substances and/or of a salt of a saturated and/or unsaturated C₈-C₂₂ fatty acid,
(f) from 0 to 50% by weight of an alcohol, and optionally
(e) from 0 to 50% by weight of typical ingredients for cleaning and disinfectant compositions and optionally
(f) tap water or deionised water ad 100%.

25. A method according to claim 24, wherein the washing liquor contains no component (a₁).

26. A method for imparting antimicrobial properties to textile fibre materials, which method comprises treating the textile fibre materials in the washing liquor with a composition comprising
(a) from 0.01 to 10% by weight of a mixture of a microbicidal active ingredient of
(a₁) a diphenyl ether compound and
(a₂) a phenol derivative,
(b) from 0 to 50% by weight of one or more hydrotropic agents,
(c) from 5 to 80% by weight of one or more synthetic detergents or of a soap or of combinations of the mentioned substances and/or of a salt of a saturated and/or unsaturated C₈-C₂₂ fatty acid,
(d) from 0 to 50% by weight of an alcohol,
(e) from 0 to 50% by weight of typical ingredients for cleaning and disinfectant compositions and optionally and optionally
(f) tap water or deionised water ad 100%,
at least a fraction of the antimicrobial active ingredient remaining on the textile fibre material.
